(19) United States
(12) Patent Application Publication
(10) Pub. No.: US 2004/0048339 A1
(43) Pub. Date: Mar. 11, 2004

(54) Human proteins having transmembrane domains and cDNAs encoding these proteins

(75) Inventors: Seishi Kato, Sagamihara-shi (JP); Shingo Sekine, Ageo-shi (JP)

Correspondence Address: FITZPATRICK CELLA HARPER & SCINTO, 30 ROCKEFELLER PLAZA, NEW YORK, NY, 10112, US

(73) Assignees: Sagami Chemical Research Center, Sagamihara-shi (JP); Protegene, Inc., Tokyo (JP)

(21) Appl. No.: 10/616,942

(22) Filed: Jul. 11, 2003

Related U.S. Application Data
(63) Continuation of application No. 09/529,100, filed on Aug. 21, 2000, now abandoned.
Application No. 09/529,100 is a 371 of international application No. PCT/JP98/04474, filed on Oct. 5, 1998.

(30) Foreign Application Priority Data
Oct. 8, 1997 (JP) 9/276269

(51) Int. Cl. C07K 14/705; C07H 21/04; C12P 21/02; C12N 5/06

(52) U.S. Cl. 435/69.1; 435/320.1; 435/325; 530/350; 536/23.5

(57) ABSTRACT

The invention provides human proteins having transmembrane domains and cDNAs coding for these proteins as well as eucaryotic cells expressing said cDNAs. All of the proteins exist in the cell membrane, so that they are considered to be proteins controlling the proliferation and the differentiation of the cells. Accordingly, the proteins can be employed as pharmaceuticals such as carcinostatic agents relating to the control of the proliferation and the differentiation of the cells or as antigens for preparing antibodies against said proteins. The cDNAs can be utilized as probes for the gene diagnosis and gene sources for the gene therapy. Furthermore, the cDNAs can be utilized for large-scale expression of said proteins. Cells, wherein these membrane protein genes are introduced and membrane proteins are expressed in large amounts, can be utilized for detection of the corresponding ligands, screening of novel low-molecular pharmaceuticals, and so on.

### TECHNICAL FIELD

The present invention relates to human proteins having transmembrane domains and cDNAs coding for these proteins as well as eucaryotic cells expressing said cDNAs. The proteins of the present invention can be employed as pharmaceuticals or as antigens for preparing antibodies against said proteins. The human cDNAs of the present invention can be utilized as probes for the gene diagnosis and gene sources for the gene therapy. Furthermore, the cDNAs can be utilized as gene sources for large-scale production of the proteins encoded by said cDNAs. Cells, wherein these membrane protein genes are introduced and membrane proteins are expressed in large amounts, can be utilized for detection of the corresponding ligands, screening of novel low-molecular pharmaceuticals, and so on.

### BACKGROUND ART

Membrane proteins play important roles, as signal receptors, ion channels, transporters, etc. in the material transportation and the information transmission which are mediated by the cell membrane. Examples thereof include receptors for a variety of cytokines, ion channels for the sodium ion, the potassium ion, the chloride ion, etc., transporters for saccharides and amino acids, and so on, where the genes of many of them have been cloned already.

It has been clarified that abnormalities of these membrane proteins are associated with a number of hitherto-cryptogenic diseases. For instance, a gene of a membrane protein having twelve transmembrane domains was identified as the gene responsible for cystic fibrosis [Rommens, J. M. et al., Science 245: 1059-1065 (1989)]. In addition, it has been clarified that several membrane proteins act as receptors when a virus infects the cells. For instance, HIV-1 is revealed to infect into the cells through mediation of a membrane protein fusion having a membrane protein on the T-cell membrane, a CD-4 antigen, and 7 transmembrane domains [Feng, Y. et al., Science 272: 872-877 (1996)]. Therefore, discovery of a new membrane protein is anticipated to lead to elucidation of the causes of many diseases, so that isolation of a new gene coding for the membrane protein has been desired.

Heretofore, owing to difficulty in the purification, many membrane proteins have been isolated by an approach from the gene side. A general method is the so-called expression cloning which comprises transfection of a cDNA library in eucaryotic cells to express cDNAs and then detection of the cells expressing the target membrane protein on the membrane by an immunological technique using an antibody or a physiological technique on the change in the membrane permeability. However, this method is applicable only to cloning of a gene of a membrane protein with a known function.

In general, membrane proteins possess hydrophobic transmembrane domains inside the proteins, wherein, after synthesis thereof in the ribosome, these domains remain in the phospholipid membrane to be trapped in the membrane. Accordingly, the evidence of the cDNA for encoding the membrane protein is provided by determination of the whole base sequence of a full-length cDNA followed by detection of highly hydrophoblc transmembrane domains in the amino acid sequence of the protein encoded by said cDNA.

### DISCLOSURE OF INVENTION

The object of the present invention is to provide novel human proteins having transmembrane domains and DNAs coding for said proteins as well as transformation eucaryotic cells that are capable of expressing said cDNAs.

As the result of intensive studies, the present inventors have been successful in cloning of cDNAs coding for proteins having transmembrane domains from the human full-length cDNA bank, thereby completing the present invention. In other words, the present invention provides human proteins having transmembrane domains, namely proteins containing any of the amino acid sequences represented by Sequence Nos. 1 to 6. Moreover, the present invention provides DNAs coding for the above-mentioned proteins, exemplified by cDNAs containing any of the base sequences represented by Sequence Nos. 7 to 12, as well as transformation eucaryotic cells that are capable of expressing said cDNAs.

### BRIEF DESCRIPTION OF DRAWINGS

FIG. 1: A figure depicting the hydrophobicity/hydrophilicity profile of the protein encoded by clone HP00936.

FIG. 2: A figure depicting the hydrophobicty/hydrophilicity profile of the protein encoded by clone HP01535.

FIG. 3: A figure depicting the hydrophobicity/hydrophilicity profile of the protein encoded by clone HP10089.

FIG. 4: A figure depicting the hydrophobicity/hydrophilicity profile of the protein encoded by clone HP10216.

FIG. 5: A figure depicting the hydrophobicity/hydrophilicity profile of the protein encoded by clone HP10420.

FIG. 6: A figure depicting the hydrophobicity/hydrophilicity profile of the protein encoded by clone HP10441.

### BEST MODE FOR CARRYING OUT THE INVENTION

The proteins of the present invention can be obtained, for example, by a method for isolation from human organs, cell lines, etc., a method for preparation of peptides by the chemical synthesis, or a method for production with the recombinant DNA technology using the DNAs coding for the transmembrane domains of the present invention, wherein the method for obtainment by the recombinant DNA technology is employed preferably. For instance, in vitro expression of the proteins can be achieved by preparation of an RNA by in vitro transcription from a vector having one of cDNAs of the present invention, followed by in vitro translation using this RNA as a template. Also, recombination of the translation region into a suitable expression vector by the method known in the art leads to production of a large amount or the encoded protein by using prokaryotic cells such as Escherichia coli, Bacillus subtilis, etc., and eucaryotic cells such as yeasts, insect cells, mammalian cells, etc.

In the case in which a protein of the present invention is produced by a microorganism such as Escherichia coli etc., a recombinant expression vector bearing the translation region in the cDNA of the present invention is constructed in an expression vector having an origin, a promoter, a ribosome-binding site, a cDNA-cloning site, a terminator etc., which can be replicated in the microorganism, and, after transformation of the host cells with said expression vector, the thus-obtained transformant is incubated, whereby the protein encoded by said cDNA can be produced on a large scale in the microorganism. In this case, a protein fragment containing an optional region can be obtained by carrying out the expression with inserting an initiation codon and a termination codon in front of and behind an optional translation region. Alternatively, a fusion protein with another protein can be expressed. Only a protein portion coding for said cDNA can be obtained by cleavage of said fusion protein with a suitable protease.

In the case in which one of the proteins of the present invention is produced in eucaryotic cells, the protein of the present invention can be produced as a transmembrane protein on the cell-membrane surface, when the translation region of said cDNA is subjected to recombination to an expression vector for eucaryotic cells that has a promoter, a splicing region, a poly(A) insertion site, etc., followed by introduction into the eucaryotic cells. The expression vector is exemplified by pKA1, pED6dp2, pCDM8, pSVK3, pMSG, pSVL, pBK-CMV, pBK-RSV, EBV vector, pRS, pYES2, and so on. Examples of eucaryotic cells to be used in general include mammalian culture cells such as simian kidney cells COS7, Chinese hamster ovary cells CHO, etc., budding yeasts, fission yeasts, silkworm cells, Xenopus laevis egg cells, and so on, but any eucaryotic cells may be used, provided that they are capable of expressing the present proteins on the membrane surface. The expression vector can be introduced in the eucaryotic cells by methods known in the art such as the electroporation method, the potassium phosphate method, the liposome method, the DEAE-dextran method, and so on.

After one of the proteins of the present invention is expressed in prokaryotic cells or eucaryotic cells, the objective protein can be isolated from the culture and purified by a combination of separation procedures known in the art. Such examples include treatment with a denaturing agent such as urea or a surface-active agent, sonication, enzymatic digestion, salting-out or solvent precipitation, dialysis, centrifugation, ultrafiltration, gel filtration, SDS-PAGE, isoelectric focusing, ion-exchange chromatography, hydrophobic chromatography, affinity chromatography, reverse phase chromatography, and so on.

The proteins of the present invention include peptide fragments (more than 5 amino acid residues) containing any partial amino acid sequence in the amino acid sequences represented by Sequence Nos. 1. to 6. These peptide fragments can be utilized as antigens for preparation of antibodies. Hereupon, among the proteins of the present invention, those having the signal sequence are secreted in the form of maturation proteins on the surface of the cells, after the signal sequences are removed. Therefore, these maturation proteins shall come within the scope of the present invention. The N-terminal amino acid sequences of the maturation proteins can be easily identified by using the method for the cleavage-site determination in a signal sequence [Japanese Patent Kokai Publication No.1996-187100]. Furthermore, some membrane proteins undergo the processing on the cell surface to be converted to the secretory forms. Such proteins or peptides in the secretory forms shall come within the scope of the present invention. When sugar chain-binding sites are present in the amino acid sequences, expression in appropriate eucaryotic cells affords proteins wherein sugar chains are added. Accordingly, such proteins or peptides wherein sugar chains are added shall come within the scope of the present invention.

The DNAs of the present invention include all DNAs coding for the above-mentioned proteins. Said DNAs can be obtained by using a method by chemical synthesis, a method by cDNA cloning, and so on.

The cDNAs of the present invention can be cloned, for example, from cDNA libraries of the human cell origin. These cDNA are synthesized by using as templates poly(A) RNAs extracted from human cells. The human cells may be cells delivered from the human body, for example, by the operation or may be the culture cells. The cDNAs can be synthesized by using any method selected from the Okayama-Berg method [Okayama, H. and Berg, P., Mol. Cell. Biol. 2: 161-170 (1982)], the Gubler-Hoffman method [Gubler, U. and Hoffman, J. Gene 25: 263-269 (1983)], and soon, but it is preferred to use the capping method [Kato, S. et al., Gene 150: 243-250 (1994)], as exemplified in Examples, in order to obtain a full-length clone in an effective manner.

The primary selection of one of the cDNAs coding for the human proteins having transmembrane domains is carried out by sequencing of a partial base sequence of a cDNA clone selected at random from cDNA libraries, sequencing of the amino acid sequence encoded by the base sequence, and recognition of the presence or absence of a hydrophobic site in the resulting N-terminal amino acid sequence region. Next, the secondary selection is carried out by determination of the whole sequence by the sequencing and the protein expression by in vitro translation. Ascertainment of cDNAs of the present invention for encoding the proteins having secretory signal sequences is carried out by using the signal sequence detection method [Yokoyama-Kobayashi, M. et al., Gene 163: 193-196 (1995)]. In other words, the ascertainment for a coding portion of an inserted cDNA fragment to function as a signal sequence is provided by fusing a cDNA fragment coding for the N-terminus of the target protein with a cDNA coding for the protease domain of urokinase and then expressing the resulting cDNA in COS7 cells to detect the urokinase activity in the cell culture medium. On the other hand, in the case in which the urokinase activity is not detectable in the cell medium, the N-terminal region is judged to remain in the membrane.

The cDNAs of the present invention are characterized by containing either of the base sequences represented by Sequence Nos. 7 to 12 or the base sequences represented by Sequence Nos. 13, 15, 17, 19, 21 and 23. Table 1 summarizes the clone number (HP number), the cells affording the cDNA, the total base number of the cDNA, and the number of the amino acid residues of the encoded protein, for each of the cDNAs.

TABLE 1

| | | | Number of | Number of |
|---|---|---|---|---|
| Sequence No. | HP No. | Cell | bases | amino acids |
| 1, 7, 13 | HP00956 | F-2 0S | 867 | 168 |
| 2, 8, 14 | HP01535 | Stomach cancer | 720 | 164 |
| 3, 9, 15 | HP10089 | Liver | 566 | 141 |
| 4, 10, 16 | HP10216 | HT-1080 | 1078 | 142 |
| 5, 11. 17 | HP10420 | Stomach cancer | 1310 | 346 |
| 6, 12, 18 | HP10441 | Stomach cancer | 781 | 66 |

Hereupon, the same clones as the cDNAs of the present invention can be easily obtained by screening of the cDNA libraries constructed from the human cell lines and human tissues utilized in the present invention by the use of an oligonucleotide probe synthesized on the basis of the cDNA base sequence described in any of Sequence Nos. 7. to 12, 13, 15, 17, 19, 21 and 23.

In general, the polymorphism due to the individual difference is frequently observed in human genes. Accordingly, any cDNA that is subjected to insertion or deletion of one or plural nucleotides and/or substitution with other nucleotides in Sequence Nos. 7 to 12, 13, 15, 17, 19, 21 and 23 shall come within the scope of the present invention.

In a similar manner, any protein that is formed by these modifications comprising insertion or deletion of one or plural amino acids and/or substitution with other amino acids shall come within the scope of the present invention, as far as the protein possesses the activity of any protein having the amino acid sequences represented by Sequence Nos. 1 to 6.

The cDNAs of the present invention include cDNA fragments (more than 10 bp) containing any partial base sequence in the base sequences represented by Sequence Nos. 7 to 12 or in the base sequences represented by Sequence Nos. 13, 15, 17, 19, 21 and 23. Also, DNA fragments consisting of a sense chain and an anti-sense chain shall come within this scope. These DNA fragments can be utilized as the probes for the gene diagnosis.

In addition to the activities and uses described above, the polynucleotides and proteins of the present invention may exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

### Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodiesusing DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation. or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

### Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feel of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

### Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137-3494-3500, 1986; Bertagnolli et al., J. Immunol. 145:1706-1712, 1990; Bertagnoli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. in Current Protocols in Immunology. J.E.e.a. Coligan eds. Vol 1 pp. 3.12.1-3.12.14, John Wiley and Sons, Toronto. 1994; and Measurement of mouse and human Interferony, Schreiber, R. D. In Current Protocols in Immunology. J. E.e.a. Coligan eds. Vol 1 pp. 6.8.1-6.8.8, John Wiley and Sons, Toronto. 1994.

Assays For proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J.E.e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6-Nordan, R. In Current Protocols in Immunology. J.E.e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11-Bennett, F., Giannotti, J., Clark, S. C. and Turner, K. J. In Current Protocols in Immunology. J.E.e.a. Coligan eds. Vol 1 pp. 6.15.1 John Wiley and Sons, Toronto. 1991; Measurement of mouse and human Interleukin 9- Ciarletta, A., Giannotti, J., Clark, S. C. and Turner, K. J. In Current Protocols in Immunology. J.E.e.a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Edby J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger etal., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

### Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in craft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligands) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to aneraize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/ipr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class IIα chain protein and an MHC class IIβ chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Edby J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., Immunol. 137:3494-3500, 1986; Bowmanet al., J. Virology 61:1992-1998; Takai et al., J. Immunol. 140:508-512, 1988; Bertagnolli et al., Cellular Immunclogy 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J.E.e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takaietal., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad Sci. USA 88:7548-7551, 1991.

### Hematoepoiesis Regulating Activity

A protein of the present invention may be useful in regulation of hematopoiesis and, consequently, in the treatment of myeloid or lymphoid cell deficiencies. Even marginal biological activity in support of colony forming cells or or factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in.: Johansson et al. Cellular Biology 15:141-151, 1995; Keller et al., Molecular and Cellular Biology 13:473-486, 1993; McClanahan et ai., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, etal. eds. Vol pp.265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Fresnney, et al. eds. Vol pp. 163-179, Wiley-Liss, Inc., New York, N.Y. 1994; Long term culture initiating cell assay, Sutherland, F. J. In Culture of Hematopoietic Cells. R. I. Freshney, et al. eds. Vol pp. 139-162, Wiley-Liss, Inc., New York, N.Y. 1994.

### Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pps. 71-112 (Maibach, H I and Rovee, D T, eds.), Year Book Medical Publishers, Inc., Chicago, as modified by Eaglstein and Mertz, J. Invest. Dermatol 71:382-84 (1978).

### Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-β group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA83:3091-3095, 1986.

### Chemotactic/Chemokinectic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis)consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

### Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolyzic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

### Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphonatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligend interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: Current Protocols in Immunlogy, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein etal., J. Exp. Med. 169:149-160 1989; Stoltenborg et al., J. Immunol. Methods 175:59-68, 1994; Stitt et al., Cell 80:661-670, 1995.

### Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of ytokines such as TNF or IL-1. Proteins of the invention may also be useful to treat anaphyiaxis and hypersensitivity to an antigenic substance or material.

### Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth

### Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or components); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

### EXAMPLES

The present invention is embodied in more detail by the following examples, but this embodiment is not intended to restrict the present invention. The basic operations and the enzyme reactions with regard to the DNA recombination are carried out according to the literature [“Molecular Cloning. A Laboratory Manual”, Cold Spring Harbor Laboratory, 1989]. Unless otherwise stated, restrictive enzymes and a variety of modification enzymes to be used were those available from TAKARA SHUZO. The manufacturer's instructions were used for the buffer compositions as well as for the reaction conditions, in each of the enzyme reactions. The cDNA synthesis was carried out according to the literature [Kato, S. et al., Gene 150: 243-250 (1994)].

(1) Preparation of Poly(A)^{·} RNA

The fibrosarcoma cell line HT-1080 (ATCC CCL 121), the osteosarcoma cell line U-2 CS (ATCC HTB 96), tissues of stomach cancer delivered by the operation, and the liver were used for human cells to extract mRNAs. The cell lines were incubated by a conventional procedure.

After about 1 g of the human cells was homogenized in 20 ml of a 5.5 M guanidinium thiocyanate solution, a total mRNA was prepared according to the literature [Okayama, H. et al., “Method in Enzymology”, Vol. 164, Academic Press, 1987]. This was subjected to chromatography on oligo (dT)-cellulose column washed with a 20 mM Tris-hydrochloride buffer solution (pH 7.6), 0.5 M NaCl, and 1 mM EDTA to obtain a poly(A)^{·} RNA according to the above-described literature.

(2) Construction of cDNA Library

Ten micrograms of the above-mentioned poly(A)^{·} RNA were dissolved in a 100 mM Tris-hydrochloride buffer solution (pH 8), one unit of an RNase-free, bacterial alkaline phosphatase was added, and the reaction was run at 37° C. for one hour. After the reaction solution was subjected to phenol extraction, followed by ethanol precipitation, the resulting pellet was dissolved in a solution containing 50 mM sodium acetate (pH 6), 1 mM EDTA, 0.1% 2-mercaptoethanol, and 0.01% Triton X-100. Thereto was added one unit of a tobacco-origin acid pyrophosphatase (Epicentre Technologies) and a total 100 μl volume of the resulting mixture was reacted at 37° C. for one hour. After the reaction solution was subjected to phenol extraction, followed by ethanol precipitation, the resulting pellet was dissolved in water to obtain a solution of a decapped poly(A)^{·} RNA.

The decapped poly(A)^{·} RNA and 3 nmol of a chimeric DNA-RNA oligonucleotide (5′-dG-dG-dG-dG-dA-dA-dT-dT-dC-dG-dA-G-G-A-3′) were dissolved in a solution containing 50 mM Tris-hydrochloride buffer solution (pH 7.5), 0.5 mM ATP, 5 mM MgCl₂, 10 mM 2-mercaptoethanol, and 25% polyethylene glycol, whereto was added 50 units of T4RNA ligase and a total 30 μl volume of the resulting mixture was reacted at 20° C. for 12 hours. After the reaction solution was subjected to phenol extraction, followed by ethanol precipitation, the resulting pellet was dissolved in water to obtain a chimeric-oligo-capped poly(A)^{·} RNA.

After digestion of vector pKA1 (Japanese Patent Kokai Publication No. 1992-117292) developed by the present inventors with KpnI, about 60 dT tails were added using a terminal transferase. A vector primer to be used below was prepared by digestion of this product with EcoRV to remove a dT tail at one side.

After 6 μg of the previously-prepared chimeric-oligo-capped poly(A)^{·} RNA was annealed with 1.2 μg of the vector primer, the resulting product was dissolved in a solution containing 50 mM Tris-hydrochloride buffer solution (pH 8.3), 75 mM KCl, 3 mM MgCl₂, 10 mM dithiothreitol, and 1.25 mM DNTP (DATP+dCTP+dGTP+dTTP), 200 units of a reverse transcriptase (GIBCO-BRL) were added, and the reaction in a total 20 μl volume was run at 42° C. for one hour. After the reaction solution was subjected to phenol extraction, followed by ethanol precipitation, the resulting pellet was dissolved in a solution containing 50 mM Tris-hydrochloride buffer solution (pH 7.5), 100 mM NaCl, 10 mM MgCl₂, and 1 mM dithiothreitol. Thereto were added 100 units of EcoRI and a total 20 μl volume of the resulting mixture was reacted at 37° C. for one hour. After the reaction solution was subjected to phenol extraction, followed by ethanol precipitation, the resulting pellet was dissolved in a solution containing 20 mM Tris-hydrochloride buffer solution (pH7.5), 100 mM KCl, 4 mM MgCl₂, 10 mM (NH₄)₂SO₂, and 50 μg/ml of the bovine serum albumin. Thereto were added 60 units of an Escherichia coli DNA ligase and the resulting mixture was reacted at 16° C. for 16 hours. To the reaction solution were added 2 μl of 2 mM dNTP, 4 units of Escherichia coli DNA polymerase I, and 0.1 unit of Escherichia coli RNase H and the resulting mixture was reacted at 12° C. for one hour and then at 22° C. for one hour.

Next, the cDNA-synthesis reaction solution was used for transformation of Escherichia coli DH12S (GIBCO-BRL). The transformation was carried out by the electroporation method. A portion of the transformant was sprayed on the 2×YT agar culture medium containing 100 μg/ml ampicillin and the mixture was incubated at 37° C. overnight. A colony formed on the agar medium was picked up at random and inoculated on 2 ml of the 2×YT culture medium containing 100 μg/ml ampicillin. After incubation at 37° C. overnight, the culture mixture was centrifuged to separate the mycelia, from which a plasmid DNA was prepared by the alkaline lysis method. The plasmid DNA was subjected to double digestion with EcoRI and NotI, followed by 0.8% agarose gel electrophoresis, to determine the size of the cDNA insert. Furthermore, using the thus-obtained plasmid as a template, the sequence reaction was carried out by using an M13 universal primer labeled with a fluorescent dye and a Taq polymerase (a kit of Applied Biosystems) and then the product was examined with a fluorescent DNA sequencer (Applied Biosystems) to determine an about 400-bp base sequence at the 5′-terminus of the cDNA. The sequence data were filed as the homo/protein cDNA bank database.

(3) Selection of cDNAs Encoding Proteins Having Transmembrane Domains

A base sequence registered in the homo/protein cDNA bank was converted to three frames of amino acid sequences and the presence or absence of an open reading frame (ORF) beginning from the initiation codon was examined. Then, the selection was made for the presence of a signal sequence that is characteristic to a secretory protein at the N-terminus of the portion encoded by the ORF. These clones were sequenced from the both 5′ and 3′ directions by the use of the deletion method using exonuclease III to determine the whole base sequence. The hydrophobicity/hydrophilicity profiles were obtained for proteins encoded by the ORF by the Kyte-Doolittle method [Kyte, J. & Doolittle, R. F., J. Mol. Biol. 157:105-132 (1982)] to examine the presence or absence of a hydrophobic region. In the case in which there is a hydrophobic region of a putative transmembrane domain in the amino acid sequence of an encoded protein, this protein was judged as a membrane protein.

(4) Functional Verification of Secretory Signal Sequence or Transmembrane Domains

It was verified by the method described in the literature [Yokoyama-Kobayashi, M. et al., Gene 163: 193-196 (1995)] that the N-terminal hydrophobic region in the secretory protein clone candidate obtained in the above-mentioned steps functions as a secretory signal sequence. First, the plasmid containing the target cDNA was cleaved at an appropriate restriction enzyme site existing at the downstream of the portion expected for encoding the secretory signal sequence. In the case in which this restriction site was a protruding terminus, the site was blunt-ended by the Klenow treatment or treatment with the mung-bean nuclease. Digestion with HindIlI was further carried out and a DNA fragment containing the SV40 promoter and a cDNA encoding the secretory signal sequence at the downstream of the promoter was separated by agarose gel electrophoresis. The resulting fragment was inserted between HindIII in pSSD3 (DDBJ/EMBL/GenBank Registration No. AB007632) and a restriction enzyme site selected so as to match with the urokinase-coding frame, thereby constructing a vector expressing a fusion protein of the secretory signal sequence of the target cDNA and the urokinase protease domain.

After Escherichia coli (host: JM109) bearing the fusion-protein expression vector was incubated at 37° C. for 2 hours in 2 ml of the 2×YT culture medium containing 100 μg/ml of ampicillin, the helper phage M13KO7 (50 μl) was added and the incubation was continued at 37° C. overnight. A supernatant separated by centrifugation underwent precipitation with polyethylene glycol to obtain single-stranded phage particles. These particles were suspended in 100 μl of 1 mM Tris-0.1 mM EDTA, pH 8 (TE). Also, there were used as controls suspensions of single-stranded phage particles prepared in the same manner from pSSD3 and from the vector pKA1-UPA containing a full-length cDNA of urokinase [Yokoyama-Kobavashi, M. et al., Gene 163: 193-196 (1995)].

The culture cells originating from the simian kidney, COS7, were incubated at 37° C. in the presence of 5% CO₂ in the Dulbecco's modified Eagle's culture medium (DMEM) containing 10% fetal calf albumin. Into a 6-well plate (Nunc Inc., 3 cm in the well diameter) were inoculated 1×10⁵ COS7 cells and incubation was carried out at 37° C. for 22 hours in the presence of 5% CO₂. After the culture medium was removed, the cell surface was washed with a phosphate buffer solution and then washed again with DMEM containing 50 mM Tris-hydrochloric acid (pH 7.5) (TDMEM). To the resulting cells was added a suspension of 1 μl of the single-stranded phage suspension, 0.6 ml of the DMEM culture medium, and 3 μl of TRANSFECTAM™ (IBF Inc.) and the resulting mixture was incubated at 37° C. for 3 hours in the presence of 5% CO₂. After the sample solution was removed, the cell surface was washed with TDMEM, 2 ml per well of DMEM containing 10% fetal calf albumin was added, and the incubation was carried out at 37° C. for 2 days in the presence of 5% CO₂.

To 10 ml of 50 mM phosphate buffer solution (pH 7.4) containing 2% bovine fibrinogen (Miles Inc.), 0.5% agarose, and 1 mM calcium chloride were added 10 units of human thrombin (Mochida Pharmaceutical Co., Ltd.) and the resulting mixture was solidified in a plate of 9 cm in diameter to prepare a fibrin plate. Ten microliters of the culture supernatant of the transfected COS7 cells were spotted on the fibrin plate, which was incubated at 37° C. for 15 hours. In the case in which a clear circle appears on the fibrin plate, it is judged that the cDNA fragment codes for the amino acid sequence functioning as a secretory signal sequence. On the other hand, in case in which a clear circle is not formed, the cells were washed well, then the fibrin sheet was placed on the cells, and incubation was carried our at 37° C. for 15 hours. In case in which a clear portion is formed on the fibrin sheet, it indicates that the urokinase activity was expressed on the cell surface. In other words, the cDNA fragment is judged to code for the transmembrane domains.

(5) Protein Synthesis By in Vitro Translation

The plasmid vector bearing the cDNA of the present invention was used for in vitro transcription/translation with a T_{N}T rabbit reticulocyte lysate kit (Promega). In this case, [³⁵S]methionine was added to label the expression product with a radioisotope. Each of the reactions was carried out according to the protocols attached to the kit. Two micrograms of the plasmid was reacted at 30° C. for 90 minutes in a total 25 μl volume of the reaction solution containing 12.51 μl of T_{N}T rabbit reticulocyte lysate, 0.5 μl of a buffer solution (attached to kit), 2 μl of an amino acid mixture (methionine-free), 2 μl of [³⁵S]methionine (Amersham) (0.37 MBq/μl), 0.5 μl of T7RNA polymerase, and 20 U of RNasin. To 3 μl of the resulting reaction solution was added 2 μl of the SDS sampling buffer (125 mM Tris-hydrochloric acid buffer, pH 6.8, 120 mM 2-mercaptoethanol, 2% SDS solution, 0.025% bromophenol blue, and 20% glycerol) and the resulting mixture was heated at 95° C. for 3 minutes and then subjected to SDS-polyacrylamide gel electrophoresis. The molecular weight of the translation product was determined by carrying out the autoradiograph.

(6) Expression by COS7

Escherichia coli bearing the expression vector of the protein of the present invention was infected with helper phage M13KO7 and single-stranded phage particles were obtained by the above-mentioned procedure. The thus-obtained phage was used for introducing each expression vector in the culture cells originating from the simian kidney, COS7. After incubation at 37° C. for 2 days in the presence of 5% CO₂, the incubation was continued for one hour in the culture medium containing [³⁵S]cystine or [³⁵S]methionine. Collection and dissolution of the cells, followed by subjecting to SDS-PAGE, allowed to observe the presence of a band corresponding to the expression product of each protein, which did not exist in the COS7 cells. For instance, HP01535, HP10216 and HP10420 produced respectively bands of 18 kDa, 20 kDa and 76 kDa in the supernatant of the culture medium. HP10441 also produced a band of 7 kDa in the membrane fraction.

(7) Clone Examples

<HP00956> (Sequence Nos. 1, 7, and 13)

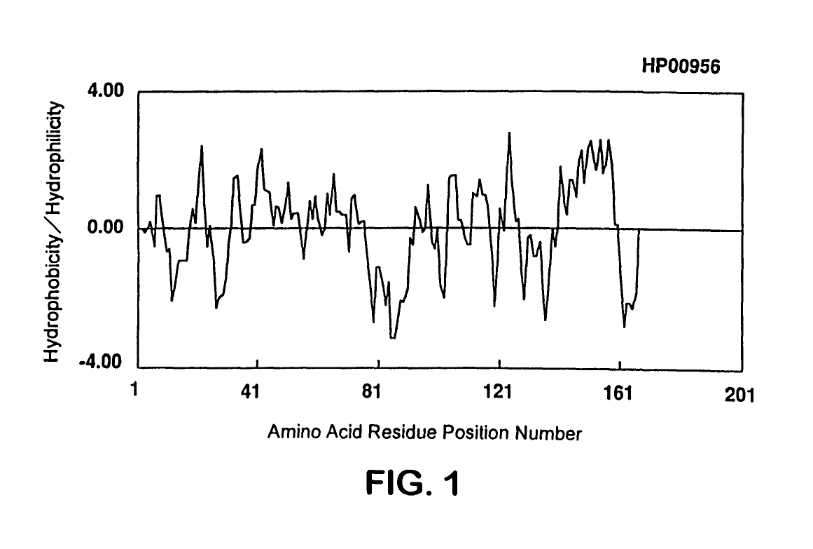

Determination of the whole base sequence of the cDNA insert of clone HP00956 obtained from cDNA libraries of the human osteosarcoma cell line revealed the structure consisting of a 211-bp 5′-nontranslation region, a 507-bp ORF, and a 149-bp 3′-nontranslation region. The ORF codes for a protein consisting of 168 amino acid residues and there existed one transmembrane domain at the C-terminal. FIG. 1 depicts the hydrophobicity/hydrophilicity profile, obtained by the Kyte-Doolittle method, of the present protein. In vitro translation resulted in formation of a translation product of 20 kDa that was almost consistent with the molecular weight of 18,750 predicted from the ORF.

The search of the protein data base by using the amino acid sequence of the present protein revealed that the protein was analogous to the human H-rev107 protein homologue (SWISS-PROT Accession No. P53816). Table 2 shows the comparison of the amino acid sequence between the human protein of the present invention (HP) and the human H-rev107 protein homologue (HR). Therein, the marks of −, *, and . represent a gap, an amino acid residue identical with the protein of the present invention, and an amino acid residue analogous to the protein of the present invention, respectively. The both proteins possessed a homology of 46.5%.

TABLE 2

| | |
|---|---|
| HS | MAFNDCFSLNYPGNPCPGDLIEVFRPGYQHWALYLGDGYVINIAPVDGIP-ASFTSAKSV |
| | .* ******.*** *.***.*.*****...** .... *. .*..*. |
| HR | MRAPIPEPKPGDLIEIFRPFYRHWAIYVGDGYVVHLAPPSEVAGAGAASVMSA |
| HS | FSSKALVKMQLLKDVVGNDTYRINNKYDETYPPLPVEEIIKRSEFVIGQEVAYNLLVNNC |
| | ...**.**..** **.*.*.*..***.*..*.*** ..**.*.* ..**** *.* .** |
| HR | LTDKAIVKKELLYDVAGSDKYQVNNKHDDKYSPLPCTKIIQRAEELVGQEVLYKLTSENC |
| HS | EHFVTLLRYGEGVSEQANRAISTVEFVTAAVGVFSFLG-LFPKGQRAKYY |
| | ****. **** . *.*.. .* ... .. .....*..* .*....*.* |
| HR | EHFVNELRYGVARSDQVRDVITAASVAGMGLAAMSLIGVMFSRNKRQKQ |

Furthermore, the search of the GenBank using the base sequences of the present cDNA has revealed the presence of sequences that possessed a homology of 90% or more (for example, Accession No. AA478132) in EST, but, since they are partial sequences, it can not be judged whether or not any of these sequences codes for the same protein as the protein of the present invention.

The human H-rev107 protein is one of proteins which are specifically expressed in H-ras resistant fibroblast cells [Hajnal, A. et al., Oncogene 9: 479-490 (1994)]. Accordingly, the present protein has been considered to be associated with phenotypic expression of cancer cells.

<HP01535> (Sequence Nos. 2, 8, and 15)

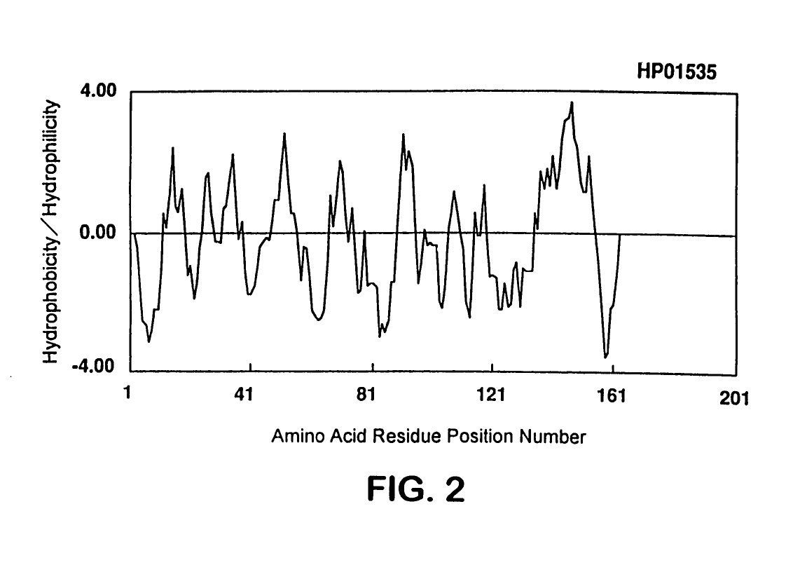

Determination of the whole base sequence of the cDNA insert of clone HP01535 obtained from cDNA libraries of human stomach cancer revealed the structure consisting of a 24-bD 5′-nontranslation region, a 495-bp ORF, and a 201-bp 3′-nontranslation region. The ORF codes for a protein consisting or 164 amino acid residues and there existed one transmembrane domain at the C-terminal. FIG. 2 depicts the hydrophobicity/hydrophilicity profile, obtained by the Kyte-Doolittle method, of the present protein. In vitro translation resulted in formation of a translation product of 20 kDa that was almost consistent with the molecular weight of 18,179 predicted from the ORF.

The search of the protein data base by using the amino acid sequence of the present protein revealed that the protein was analogous to the human H-rev107 protein homologue (SWISS-PROT Accession No. P53816). Table 3 shows the comparison of the amino acid sequence between the human protein of the present invention (HP) and the human H-rev107 protein homologue (HR). Therein, the marks of −, *, and . represent a gap, an amino acid residue identical with the protein of the present invention, and an amino acid residue analogous to the protein of the present invention, respectively. Both proteins possessed a homology of 51.2% in the entire region.

TABLE 3

| | |
|---|---|
| HS | MASPHQEPKPGDLIEIFRLGYEHWALYIGDGYVIHLAPPSEYPGAGSSSVFSVLSNSAEV |
| | *.*.************** * ***.*.*****.******* .***..**.*.*...* * |
| HR | MRAPIPEPKPGDLIEIFRPFYRHWAIYVGDGYVVHLAPPSEVAGAGAASVMSALTDKAIV |
| HS | KRERLEDVVGGCCYRVNNSLDHEYQPRPVEVIISSAKEMVGQKMKYSIVSRNCEHFVTQL |
| | *.* * **.*. *.***. *..* * * . ** .*.*.***.. *...* ******..* |
| HR | KKELLYDVAGSDKYQVNNKHDDKYSPLPCTKIIQRAEELVGQEVLYKLTSENCEHFVNEL |
| HS | RYGKSRCKQVEKAKVEVGVAT-ALGILVVAGCSFAIRRYQKKATA |
| | *** .*..** .. ....**. .*. . . * *. .. **. |
| HR | RYGVARSDQVRDVIIAASVAGMGLAAMSLIGVMFSRNKRQKQ |

Furthermore, the search of the GenBank using the base sequences of the present cDNA has revealed the presence of sequences that possessed a homology of 90% or more (for example, Accession No. R55646) in EST, but, since they are partial sequences, it can not be judged whether or not any of these sequences codes for the same protein as the protein of the present invention.

The human H-rev107 protein is one of proteins which are specifically expressed in H-ras resistant fibroblast cells [Hajnal, A. et al., Oncogene 9: 479-490 (1994)]. Accordingly, the present protein has been considered to be associated with phenotypic expression of cancer cells.

<HP10089> (Sequence Nos. 3, 9, and 17)

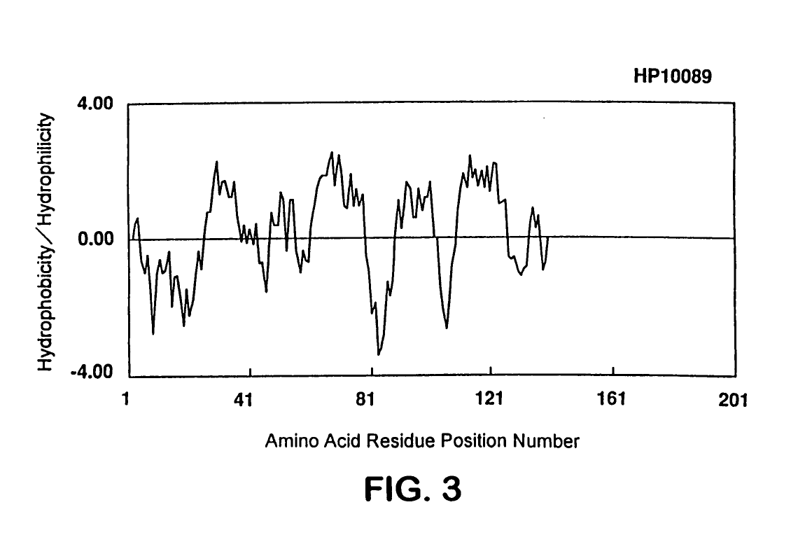

Determination of the whole base sequence of the cDNA insert of clone HP10089 obtained from cDNA libraries of human liver revealed the structure consisting of a 73-bp 5′-nontranslation region, a 426-bp ORF, and a 67-bp 3′-nontranslation region. The ORF codes for a protein consisting of 141 amino acid residues and there existed four transmembrane domains. FIG. 3 depicts the hydrophobicity/hydrophilicity profile, obtained by the Kyte-Doolittle method, of the present protein. In vitro translation resulted in formation of a translation product of 14 kDa that was almost consistent with the molecular weight of 14,852 predicted from the ORF.

The search of the protein data base using the amino acid sequence of the present protein has not revealed the presence of any known protein having an analogy. Also, the search of the GenBank using the base sequences of the present cDNA has revealed the presence of sequences that possessed a homology of 90% or more and contained an initiation codon (for example, Accession No. N56722) in EST, but, since they are partial sequences, it can not be judged whether or not any of these sequences codes for the same protein as the protein of the present invention.

<HP10216> (Sequence Nos. 4, 10, and 19)

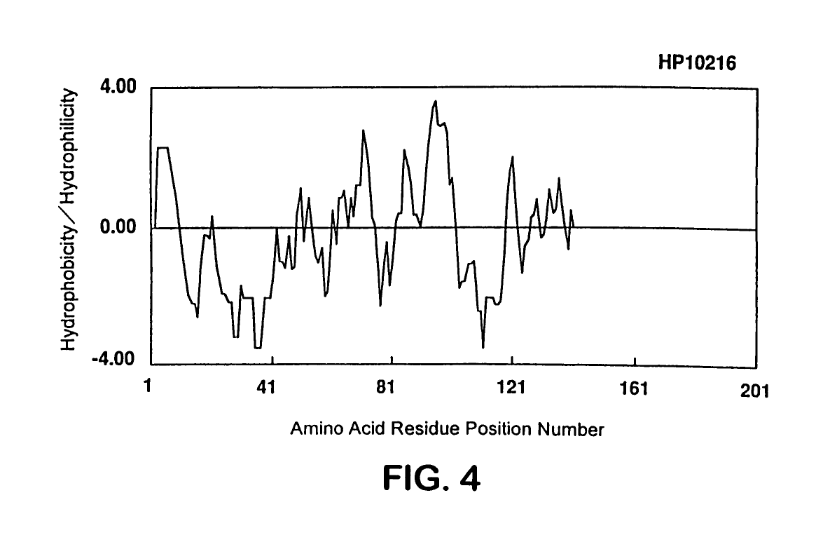

Determination of the whole base sequence of the cDNA insert of clone HP10216 obtained from cDNA libraries of the human fibrosarcoma cell line HT-1080 revealed the structure consisting of a 4-bp 5′-nontranslation region, a 429-bp ORF, and a 645-bp 3′-nontranslation region. The ORF codes for a protein consisting of 142 amino acid residues and there existed one transmembrane domain. FIG. 4 depicts the hydrophobicity/hydrophilicity profile, obtained by the Kyte-Doolittle method, of the present protein. In vitro translation resulted in formation of a translation product of 22 kDa that was larger than the molecular weight of 15,521 predicted from the ORF.

The search of the protein data base using the amino acid sequence of the present protein has not revealed the presence of any known protein having an analogy. Also, the search of the GenBank using the base sequences of the present cDNA has revealed the presence of sequences that possessed a homology of 90% or more and contained an initiation codon (for example, Accession No. AA316462) in EST, but any of the sequences was shorter than the present cDNAs and was not found to contain the initiation codon.

<HP10420> (Sequence Nos. 5, 11, and 21)

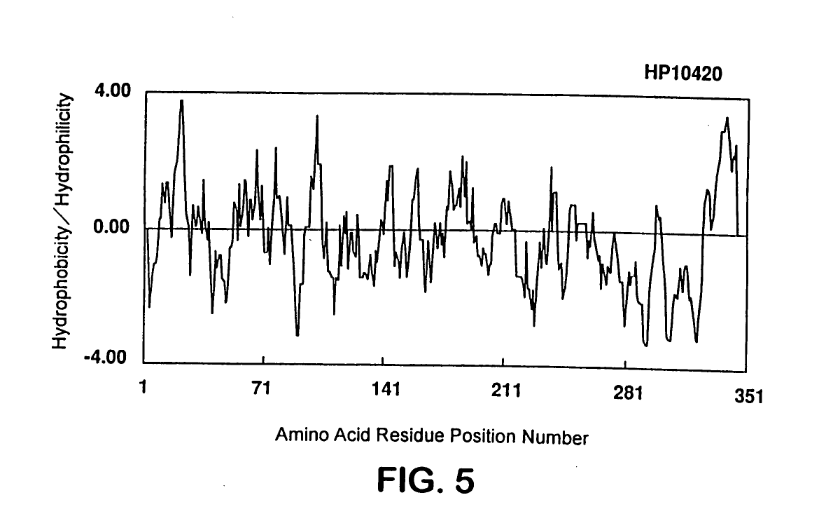

Determination of the whole base sequence of the cDNA insert of clone HP10420 obtained from cDNA libraries of human stomach cancer revealed the structure consisting of an 81-bp 5′-nontranslation region, a 1041-bp ORF, and a 188-bp 3′-nontranslation region. The ORF codes for a protein consisting of 346 amino acid residues and there existed a signal-like sequence at the N-terminus and one transmembrane domain at the C-terminus. FIG. 5 depicts the hydrophobicity/hydrophilicity profile, obtained by the Kyte-Doolittle method, of the present protein. Introduction of an expression vector, wherein the HindIII-PvuII fragment containing a cDNA portion coding for the N-terminal 34 amino acid residues of the present protein was inserted into the HindIII-SmaI site of pSSD3, into the COS7 cells revealed the urokinase activity in the culture medium to indicate that the present protein is a type-I membrane protein. In vitro translation resulted in formation of a translation product of 39 kDa that was almost consistent with the molecular weight of 35,970 predicted from the ORF. Application of the (−3, −1) rule, a method for predicting the cleavage site in the secretory signal sequence, allows to expect that the maturation protein starts from leucine at position 31.

The search of the protein data base using the amino acid sequence of the present protein has not revealed the presence or any known protein having an analogy. Also, the search of the GenBank using the base sequences of the present cDNA has revealed the presence of sequences that possessed a homology of 90% or more and contained an initiation codon (for example, Accession No. T70513) in EST, but, since they are partial sequences, it can not be judged whether or not any of these sequences codes for the same protein as the protein of the present invention.

<HP10441> (Sequence Nos. 6, 12, and 23)

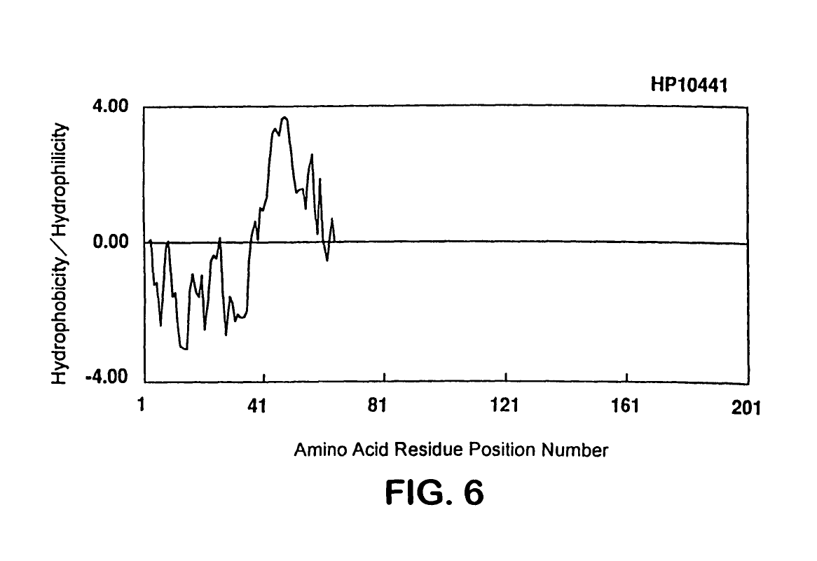

Determination of the whole base sequence of the cDNA insert of clone HP10441 obtained from cDNA libraries of the human stomach cancer revealed the structure consisting of a 341-bp 5′-nontranslation region, a 201-bp ORF, and a 239-bp 3′-nontranslation region. The ORF codes for a protein consisting of 66 amino acid residues and there existed one transmembrane domain at the C-terminus. FIG. 6 depicts the hydrophobicity/hydrophilicity profile, obtained by the Kyte-Doolittle method, of the present protein. In vitro translation resulted in formation of a translation product of 10 kDa that was almost consistent with the molecular weight of 7,374 predicted from the ORF.

The search of the protein data base using the amino acid sequence of the present protein has revealed the presence of sequences that were analogous to the nematode putative protein F59F4.2 (GenBank Accession No. Z81095). Table 4 shows the comparison of the amino acid sequence between the human protein of the present invention (HP) and the nematode putative protein F59F4.2 (CE). Therein, the marks of −, *, and . represent a gap, an amino acid residue identical with the protein of the present invention, and an amino acid residue analogous to the protein of the present invention, respectively. The both proteins possessed a homology of 52.3% in the entire region.

TABLE 4

| | |
|---|---|
| HS | MVAKQRIRMANEKHSKNITQRGNVAKTSRNAPEEKASVGPWLLALFIFVVCGSAIFQIIQ |
| | *..***. .**.. ***...******. . ..*.* ...***..**.*******.*.**. |
| CE | MAPKQRMTLANKQFSKNVNNRGNVAKSLK-PAEDKYPAAPWLIGLFVFVVCGSVFEIIR |
| HS | SIRMGM |
| | ..** |
| CE | YVKMG |

Furthermore, the search of the GenBank using the base sequences of the present cDNA has revealed the presence of sequences that possessed a homology of 90% or more and contained the initiation codon. (for example, Accession No. AA232459) in EST, but many sequences were not distinct and the same ORF as that in the present cDNA was not found.

### INDUSTRIAL APPLICABILITY

The present invention provides human proteins having transmembrane domains and cDNAs coding for these proteins as well as eucaryotic cells expressing said cDNAs. All of the proteins of the present invention exist in the cell membrane, so that they are considered to be proteins controlling the proliferation and the differentiation of the cells. Accordingly, the proteins of the present invention can be employed as pharmaceuticals such as carcinostatic agents relating to the control of the proliferation and the differentiation of the cells or as antigens for preparing antibodies against said proteins. The cDNAs of the present invention can be utilized as probes for the gene diagnosis and gene sources for the gene therapy. Furthermore, the cDNAs can be utilized for large-scale expression of said proteins. Cells, wherein these membrane protein genes are introduced and membrane proteins are expressed in large amounts, can be utilized for detection of the corresponding ligands, screening of novel low-molecular pharmaceuticals, and so on.

The present invention also provides genes corresponding the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464B1, incorporated by reference herein). In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14 (9): 629-633; Zwaal et al., 1993, Proc. Natl. Acad. Sci. USA 90(16): 7431-7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development or assay systems for the identification of molecules that interact with the protein product(s) of the corresponding gene(s).

Where the protein of the present invention is membrane-bound (e.g., is a receptor), the present invention also provides for soluble forms of such protein. In such forms part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information.

Proteins and protein fragments of the present invention include proteins with amino acid sequence lengths that are at least 25% (more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

Species homologs of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide, as determined by those of still in the art. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally-occurring alternative forms of the isolated polynucleotide which also encode proteins which are identical, homologous, or related to that encoded by the polynucleotides.

The invention also includes polynucleotides with sequences complementary to those of the polynucleotides disclosed herein.

The present invention also includes polynucleotides capable of hybridizing under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 5

| | | Hybrid | | Wash |
|---|---|---|---|---|
| Stringency | Polynucleotide | Length | Hybridization Temperature | Temperature |
| Condition | Hybrid | (bp)^{‡} | and Buffer^{†} | and Buffer^{†} |
| A | DNA:DNA | ≧50 | 65° C.: 1 × SSC -or- | 65° C.: 0.3 × SSC |
| | | | 42° C.: 1 × SSC · 50% formamide | |
| B | DNA:DNA | <50 | T_{A}*: 1 × SSC | T_{B}*: 1 × SSC |
| C | DNA:RNA | ≧50 | 67° C.: 1 × SSC -or- | 67° C.: 0.3 × SSC |
| | | | 45° C.: 1 × SSC · 50% formamide | |
| D | DNA:RNA | <50 | T_{D}*: 1 × SSC | T_{D}*: 1 × SSC |
| E | RNA:RNA | ≧50 | 70° C.: 1 × SSC -or- | 70° C.: 0.3 × SSC |
| | | | 50° C.: 1 × SSC · 50% formamide | |
| F | RNA:RNA | <50 | T_{F}*: 1 × SSC | T_{F}*: 1 × SSC |
| G | DNA:DNA | ≧50 | 65° C.: 4 × SSC -or- | 65° C.: 1 × SSC |
| | | | 42° C.: 4 × SSC · 50% formamide | |
| H | DNA:DNA | <50 | T_{H}*: 4 × SSC | T_{H}*: 4 × SSC |
| I | DNA:RNA: | ≧50 | 67° C.: 4 × SSC -or- | 67° C.: 1 × SSC |
| | | | 45° C.: 4 × SSC · 50% formamide | |
| J | DNA:RNA | <50 | T_{J}*: 4 × SSC | T_{J}*: 4 × SSC |
| K | RNA:RNA | ≧50 | 70° C.: 4 × SSC -or- | 67° C.: 1 × SSC |
| | | | 50° C.: 4 × SSC · 50% formamide | |
| L | RNA:RNA | <50 | T_{L}*: 2 × SSC | T_{L}*: 2 × SSC |
| M | DNA:DNA | ≧50 | 50° C.: 4 × SSC -or- | 50° C.: 2 × SSC |
| | | | 40° C.: 6 × SSC · 50% formamide | |
| N | DNA:DNA | <50 | T_{N}*: 6 × SSC | T_{N}*: 6 × SSC |
| O | DNA:RNA | ≧50 | 55° C.: 4 × SSC -or- | 55° C.: 2 × SSC |
| | | | 42° C.: 6 × SSC · 50% formamide | |
| P | DNA:RNA | <50 | T_{P}*: 6 × SSC | T_{P}*: 6 × SSC |
| Q | RNA:RNA | ≧50 | 60° C.: 4 × SSC -or- | 60° C.: 2 × SSC |
| | | | 45° C.: 6 × SSC · 50% formamide | |
| R | RNA:RNA | <50 | T_{R}*: 4 × SSC | T_{R}*: 4 × SSC |
| ^{‡}The hybrid length is that anticipated for the hybridized region(s) of the hybridizing polynucleotides. When hybridizing a polynucleotide to a target polynucleotide of unknown sequence. the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{†}SSPE (1 × SSPE is 0.15 M NaCl. 10 mM NaH₂PO₄, and 1.25 mM EDTA. pH7.4) can be substituted for SSC (1 × SSC is 0.15 M NaCl and 15 mM sodium citrate) in the hybridization and wash buffers: washes are performed for 15 minutes after hybridization is complete. | | | | |
| *T_{B}-T_{R}: The hybridization temperature for hybrids anticipated to be less than 50 base pairs in length should be 5-10° C. less than the melting temperature (Tₘ) of the hybrid. where Tₘ is determined according to the following equations. For hybrids less than 18 base pairs in length. Tₘ(° C.) = 2(#of A + T bases) + 4(# of G + C bases). | | | | |
| # For hybrids between 18 and 49 base pairs in length. Tₘ(° C.) = 81.5 + 16.6(log₁₀[Na⁺]) + 0.41 (%G + C) − (600/N), where N is the number of bases in the hybrid, and [Na⁺] is the concentration of sodium ions in the hybridization buffer ([Na⁺] for 1 × SSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

1. A protein comprising any of the amino acid sequences represented by Sequence Nos. 1 to 6.

2. A DNA coding for the protein according to claim 1.

3. A cDNA comprising any of the base sequences represented by Sequence Nos. 7 to 12.

4. The cDNA according to claim 3 comprising any of the base sequences represented by Sequence Nos. 13, 15, 17, 19, 21 and 23.

5. A vector capable of expressing the DNA or cDNA according to any of claims 2 to 4 in in vitro translation or an eucaryotic cell.

6. A transformation eucaryotic cell capable of expressing the DNA or according to any of claims 2 to 4 to produce the protein according to claim 1.


SEQUENCE LISTING (28 sequences)

SEQ ID NO: 1 - protein, 168 aa, Homo sapiens
MAFNDCFSLNYPGNPCPGDLIEVFRPGYQHWALYLGDGYVINIAPVDGIPASFTSAKSVFSSKALVKMQLLKDVVGNDTYRINNKYDETYPPLPVEEIIKRSEFVIGQEVAYNLLVNNCEHFVTLLRYGEGVSEQANRAISTVEFVTAAVGVFSFLGLFPKGQRAKYY

SEQ ID NO: 2 - protein, 164 aa, Homo sapiens
MASPHQEPKPGDLIEIFRLGYEHWALYIGDGYVIHLAPPSEYPGAGSSSVFSVLSNSAEVKRERLEDVVGGCCYRVNNSLDHEYQPRPVEVIISSAKEMVGQKMKYSIVSRNCEHFVTQLRYGKSRCKQVEKAKVEVGVATALGILVVAGCSFAIRRYQKKATA

SEQ ID NO: 3 - protein, 141 aa, Homo sapiens
MAPKVFRQYWDIPDGTDCHRKAYSTTSIASVAGLTAAAYRVTLNPPGTFLEGVAKVGQYTFTAAAVGAVFGLTTCISAHVREKPDDPLNYFLGGCAGGLTLGARTHNYGIGAAACVYFGIAASLVKMGRLEGWEVFAKPKV

SEQ ID NO: 4 - protein, 142 aa, Homo sapiens
MAAAVAAAGAGEPQSPDELLPKGDAEKPEEELEEDDDEELDETLSERLWGLTEMFPERVRSAAGATFDLSLFVAQKMYRFSRAALWIGTTSFMILVLPVVFETEKLQMEQQQQLQQRQILLGPNTGLSGGMPGALPSLPGKI

SEQ ID NO: 5 - protein, 346 aa, Homo sapiens [sequence omitted]

SEQ ID NO: 6 - protein, 66 aa, Homo sapiens
MVAKQRIRMANEKHSKNITQRGNVAKTSRNAPEEKASVGPWLLALFIFVVCGSAIFQIIQSIRMGM

SEQ ID NO: 7 - DNA, 504 bp, Homo sapiens
atggcgtttaatgattgcttcagtttgaactaccctggcaacccctgcccaggggacttgatcgaagtgttccgtcctggctatcagcactgggccctgtacttgggtgatggttacgttatcaacatagcacctgtagatggcattcctgcgtcctttacaagcgccaagtctgtattcagcagtaaggccctggtgaaaatgcagctcttgaaggatgttgtgggaaatgacacatacagaataaacaataaatacgatgaaacgtacccccctctccctgtggaagaaatcataaagcggtcagagtttgtaattggacaggaggtggcctataacttacttgtcaacaactgtgaacattttgtgacattgcttcgctatggagaaggagtttcagagcaggccaaccgagcgataagtaccgttgagtttgtgacagctgctgttggtgtcttctcattcctgggcttgtttccaaaaggacaaagagcaaaatactat

SEQ ID NO: 8 - DNA, 492 bp, Homo sapiens
atggcttcgccacaccaagagcccaaacctggagacctgattgagattttccgccttggctatgagcactgggccctgtatataggagatggctacgtgatccatctggctcctccaagtgagtaccccggggctggctcctccagtgtcttctcagtcctgagcaacagtgcagaggtgaaacgggagcgcctggaagatgtggtgggaggctgttgctatcgggtcaacaacagcttggaccatgagtaccaaccacggcccgtggaggtgatcatcagttctgcgaaggagatggttggtcagaagatgaagtacagtattgtgagcaggaactgtgagcactttgtcacccagctgagatatggcaagtcccgctgtaaacaggtggaaaaggccaaggttgaagtcggtgtggccacggcgcttggaatcctggttgttgctggatgctcttttgcgattaggagataccaaaaaaaagcgacagcc

SEQ ID NO: 9 - DNA, 423 bp, Homo sapiens
atggcgccgaaggtttttcgtcagtactgggatatccccgatggcaccgattgccaccgcaaagcctacagcaccaccagtattgccagcgtcgctggcctgaccgccgctgcctacagagtcacactcaatcctccgggcaccttccttgaaggagtggctaaggttggacaatacacgttcactgcagctgctgtcggggccgtgtttggcctcaccacctgcatcagcgcccatgtccgcgagaagcccgacgaccccctgaactacttcctcggtggctgcgccggaggcctgactctgggagcacgcacgcacaactacgggattggcgccgccgcctgcgtgtactttggcatagcggcctccctggtcaagatgggccggctggagggctgggaggtgtttgcaaaacccaaggtg

SEQ ID NO: 10 - DNA, 426 bp, Homo sapiens
atggctgccgccgtcgctgctgccggtgcaggggaaccccagtccccggacgaattgctcccgaaaggcgacgcggagaagcctgaggaggagctggaggaggacgacgatgaggagctagatgagaccctgtcggagagactatggggcctgacggagatgtttccggagagggtccggtccgcggccggagccacttttgatctttccctctttgtggctcagaaaatgtacaggttttccagggcagccttgtggattgggaccacttcctttatgatcctggttcttcccgttgtctttgagacggagaagttgcaaatggagcaacagcagcaactgcagcagcggcagatacttctaggacctaacacagggctctcaggaggaatgccaggggctctaccctcacttcctggaaagatc

SEQ ID NO: 11 - DNA, 1038 bp, Homo sapiens
atggaccccgccaggaaagcaggtgcccaggccatgatctggactgcaggctggctgctgctgctgctgcttcgcggaggagcgcaggccctggagtgctacagctgcgtgcagaaagcagatgacggatgctccccgaacaagatgaagacagtgaagtgcgcgccgggcgtggacgtctgcaccgaggccgtgggggcggtggagaccatccacggacaattctcgctggcagtgcggggttgcggttcgggactccccggcaagaatgaccgcggcctggatcttcacgggcttctggcgttcatccagctgcagcaatgcgctcaggatcgctgcaacgccaagctcaacctcacctcgcgggcgctcgacccggcaggtaatgagagtgcatacccgcccaacggcgtggagtgctacagctgtgtgggcctgagccgggaggcgtgccagggtacatcgccgccggtcgtgagctgctacaacgccagcgatcatgtctacaagggctgcttcgacggcaacgtcaccttgacggcagctaatgtgactgtgtccttgcctgtccggggctgtgtccaggatgaattctgcactcgggatggagtaacaggcccagggttcacgctcagtggctcctgttgccaggggtcccgctgtaactctgacctccgcaacaagacctacttctcccctcgaatcccaccccttgtccggctgccccctccagagcccacgactgtggcctcaaccacatctgtcaccacttctacctcggccccagtgagacccacatccaccaccaaacccatgccagcgccaaccagtcagactccgagacagggagtagaacacgaggcctcccgggatgaggagcccaggttgactggaggcgccgctggccaccaggaccgcagcaattcagggcagtatcctgcaaaaggggggccccagcagccccataataaaggctgtgtggctcccacagctggattggcagcccttctgttggccgtggctgctggtgtcctactg

SEQ ID NO: 12 - DNA, 198 bp, Homo sapiens
atggtcgccaagcaaaggatccgtatggccaacgagaagcacagcaagaacatcacccagcgcggcaacgtcgccaagacctcgagaaatgcccccgaagagaaggcgtctgtaggaccctggttattggctctcttcatttttgttgtctgtggttctgcaattttccagattattcaaagtatcaggatgggcatg

SEQ ID NO: 13 - DNA, 867 bp, Homo sapiens
  CDS (212)..(715)
cgtttcagcgtggcggcgctggtgctggcgttggccctggaggacggccccgagtgatggctggcgcctgcctcccgggtgtctcccgggtacagatggagtcgtcccgcggccgccggcggcaaggtcggcagctgcgaggccaagagagaccccaggacacacacagctgcctcccggtgcgagaagaagaccccggcttgagagtgagatggcgtttaatgattgcttcagtttgaactaccctggcaacccctgcccaggggacttgatcgaagtgttccgtcctggctatcagcactgggccctgtacttgggtgatggttacgttatcaacatagcacctgtagatggcattcctgcgtcctttacaagcgccaagtctgtattcagcagtaaggccctggtgaaaatgcagctcttgaaggatgttgtgggaaatgacacatacagaataaacaataaatacgatgaaacgtacccccctctccctgtggaagaaatcataaagcggtcagagtttgtaattggacaggaggtggcctataacttacttgtcaacaactgtgaacattttgtgacattgcttcgctatggagaaggagtttcagagcaggccaaccgagcgataagtaccgttgagtttgtgacagctgctgttggtgtcttctcattcctgggcttgtttccaaaaggacaaagagcaaaatactattaacaatttaccaaagagatattgatattgaaggaatttgggaggaggaaaagaaacctggggtgaatacttattttcagtgcatcattactgttccagattcctatgatggatggcagactctttaataaattgcttactgatattatctt

SEQ ID NO: 14 - protein, 168 aa, Homo sapiens; identical to SEQ ID NO: 1

SEQ ID NO: 15 - DNA, 720 bp, Homo sapiens
  CDS (25)..(516)
accagacctcctcttggcttcgagatggcttcgccacaccaagagcccaaacctggagacctgattgagattttccgccttggctatgagcactgggccctgtatataggagatggctacgtgatccatctggctcctccaagtgagtaccccggggctggctcctccagtgtcttctcagtcctgagcaacagtgcagaggtgaaacgggagcgcctggaagatgtggtgggaggctgttgctatcgggtcaacaacagcttggaccatgagtaccaaccacggcccgtggaggtgatcatcagttctgcgaaggagatggttggtcagaagatgaagtacagtattgtgagcaggaactgtgagcactttgtcacccagctgagatatggcaagtcccgctgtaaacaggtggaaaaggccaaggttgaagtcggtgtggccacggcgcttggaatcctggttgttgctggatgctcttttgcgattaggagataccaaaaaaaagcgacagcctgaagcagccacaaaatcctgtgttagaagcagctgtgggggtcccagtggagatgagcctcccccatgcctccagcagcctgaccctcgtgccctgtctcaggcgttctctagatcctttcctctgtttccctctctcgctggcaaaagtatgatctaattgaaacaagactgaaggatcaataaacagccatctgccccttc

SEQ ID NO: 16 - protein, 164 aa, Homo sapiens; identical to SEQ ID NO: 2

SEQ ID NO: 17 - DNA, 566 bp, Homo sapiens
  CDS (74)..(496)
gatagccagccgcggctgcccttgcgcttcccgagctggcggggtccgtggtgcgggatcgagattgcgggctatggcgccgaaggtttttcgtcagtactgggatatccccgatggcaccgattgccaccgcaaagcctacagcaccaccagtattgccagcgtcgctggcctgaccgccgctgcctacagagtcacactcaatcctccgggcaccttccttgaaggagtggctaaggttggacaatacacgttcactgcagctgctgtcggggccgtgtttggcctcaccacctgcatcagcgcccatgtccgcgagaagcccgacgaccccctgaactacttcctcggtggctgcgccggaggcctgactctgggagcacgcacgcacaactacgggattggcgccgccgcctgcgtgtactttggcatagcggcctccctggtcaagatgggccggctggagggctgggaggtgtttgcaaaacccaaggtgtgagccctgtgcctgccgggacctccagcctgcagaatgcgtccagaaataaattctgtgtctgtgtgtg

SEQ ID NO: 18 - protein, 141 aa, Homo sapiens; identical to SEQ ID NO: 3

SEQ ID NO: 19 - DNA, 1078 bp, Homo sapiens
  CDS (5)..(430)
agtcatggctgccgccgtcgctgctgccggtgcaggggaaccccagtccccggacgaattgctcccgaaaggcgacgcggagaagcctgaggaggagctggaggaggacgacgatgaggagctagatgagaccctgtcggagagactatggggcctgacggagatgtttccggagagggtccggtccgcggccggagccacttttgatctttccctctttgtggctcagaaaatgtacaggttttccagggcagccttgtggattgggaccacttcctttatgatcctggttcttcccgttgtctttgagacggagaagttgcaaatggagcaacagcagcaactgcagcagcggcagatacttctaggacctaacacagggctctcaggaggaatgccaggggctctaccctcacttcctggaaagatctagattgttattgctgtttgagctgtctcagtgggataagtttgaaattcaagtgtttgaactgctgataatttggatttttttttttttttttaactttggcacattgatctatctaaacccggtggggagaattatccccacattgtctcatggaaagactcaacttgcaactgtgccctccacactatccttacttctgtctccactctgataccagagtgcagccatgcagacggttattccagctctggtcacccgactcctttcaccaaattgctcctaactggaagatctcactttccccttgtggggtaggaaccgatgccagtgggagggatgtgcccctgaccattaacgactgttttttttttttttttttaaagaatggagttgttggggcgggacatgcacacaatgtgaaacagacaaaatgcattacacctgtagtgtaaagtggccactatgaatccctatgtatgagaggagggaggcaggctgcagcttcagccacagaatggggactatggaagacagcaggagctcatttcctctgcacatttcggctgttagacctgtgtgtgtgtttaaaaaaagagaagtcagtgctcactttttgtatttaaatattaaaaatgattccaactg

SEQ ID NO: 20 - protein, 142 aa, Homo sapiens; identical to SEQ ID NO: 4

SEQ ID NO: 21 - DNA, 1310 bp, Homo sapiens
  CDS (82)..(1119)
actcatcctgggctcaggtaagagggcccgagctcggaggcggcacatccaggggggacgccaagggagcaggacggagccatggaccccgccaggaaagcaggtgcccaggccatgatctggactgcaggctggctgctgctgctgctgcttcgcggaggagcgcaggccctggagtgctacagctgcgtgcagaaagcagatgacggatgctccccgaacaagatgaagacagtgaagtgcgcgccgggcgtggacgtctgcaccgaggccgtgggggcggtggagaccatccacggacaattctcgctggcagtgcggggttgcggttcgggactccccggcaagaatgaccgcggcctggatcttcacgggcttctggcgttcatccagctgcagcaatgcgctcaggatcgctgcaacgccaagctcaacctcacctcgcgggcgctcgacccggcaggtaatgagagtgcatacccgcccaacggcgtggagtgctacagctgtgtgggcctgagccgggaggcgtgccagggtacatcgccgccggtcgtgagctgctacaacgccagcgatcatgtctacaagggctgcttcgacggcaacgtcaccttgacggcagctaatgtgactgtgtccttgcctgtccggggctgtgtccaggatgaattctgcactcgggatggagtaacaggcccagggttcacgctcagtggctcctgttgccaggggtcccgctgtaactctgacctccgcaacaagacctacttctcccctcgaatcccaccccttgtccggctgccccctccagagcccacgactgtggcctcaaccacatctgtcaccacttctacctcggccccagtgagacccacatccaccaccaaacccatgccagcgccaaccagtcagactccgagacagggagtagaacacgaggcctcccgggatgaggagcccaggttgactggaggcgccgctggccaccaggaccgcagcaattcagggcagtatcctgcaaaaggggggccccagcagccccataataaaggctgtgtggctcccacagctggattggcagcccttctgttggccgtggctgctggtgtcctactgtgagcttctccacctggaaatttccctctcacctacttctctggccctgggtacccctcttctcatcacttcctgttcccaccactggactgggctggcccagcccctgtttttccaacattccccagtatccccagcttctgctgcgctggtttgcggctttgggaaataaaataccgttgtatatattc

SEQ ID NO: 22 - protein, 346 aa, Homo sapiens [sequence omitted]

SEQ ID NO: 23 - DNA, 781 bp, Homo sapiens
  CDS (342)..(539)
caattcccgttgttgcgttgcgtttccttcctctttcactccgcgctcacggcggcggccaaagcggcggcgacggcggcgcgagaacgacccggcggccagttctcttcctcctgcgcacctgccctgctcggtcagtcagtcggcggccggcgcccggcttgtgctcagacctcgcgcttgcggcgcccaggcccagcggccgtagctagcgtctggcctgagaacctcggcgctccggcggcgcgggcaccacgagcggagcctcgcagcggctccagaggaggcaggcgagtgagcgagtccgaggggtggccggggcaggtggtggcgccgcgaagatggtcgccaagcaaaggatccgtatggccaacgagaagcacagcaagaacatcacccagcgcggcaacgtcgccaagacctcgagaaatgcccccgaagagaaggcgtctgtaggaccctggttattggctctcttcatttttgttgtctgtggttctgcaattttccagattattcaaagtatcaggatgggcatgtgaagtgactgaccttaagatgtttccattctcctgtgaattttaacttgaactcattcctgatgtttgataccctggttgaaaacaattcagtaaagcatcctgcctcagaatgactttcctatcatgcttcatgtgtcattccaaggtttcttcatgagtcattccaagttttctagtccataccacagtgccttgcaaaaaacaccacatgaataaagcaataaaatttgattgttaag

SEQ ID NO: 24 - protein, 66 aa, Homo sapiens; identical to SEQ ID NO: 6

SEQ ID NO: 25 - DNA, 14 bp, Artificial Sequence
  Description of Artificial Sequence chimeric DNA-RNA oligonucleotide
ggggaattcgagga

SEQ ID NO: 26 - protein, 162 aa, Homo sapiens
MRAPIPEPKPGDLIEIFRPFYRHWAIYVGDGYVVHLAPPSEVAGAGAASVMSALTDKAIVKKELLYDVAGSDKYQVNNKHDDKYSPLPCTKIIQRAEELVGQEVLYKLTSENCEHFVNELRYGVARSDQVRDVIIAASVAGMGLAAMSLIGVMFSRNKRQKQ

SEQ ID NO: 27 - protein, 162 aa, Homo sapiens; identical to SEQ ID NO: 26

SEQ ID NO: 28 - protein, 64 aa, Nematode
MAPKQRMTLANKQFSKNVNNRGNVAKSLKPAEDKYPAAPWLIGLFVFVVCGSAVFEIIRYVKMG